(19) United States
(12) Patent Application Publication
(10) Pub. No.: US 2023/0329612 A1
(43) Pub. Date: Oct. 19, 2023

(54) DETERMINING DRIVER CAPABILITY

(71) Applicant: Micron Technology, Inc., Boise, ID (US)

(72) Inventors: Lisa R. Copenspire-Ross, Boise, ID (US); Nkiruka Christian, Bristow, VA (US); Trupti D. Gawai, Boise, ID (US); Josephine T. Hamada, Folsom, CA (US); Anda C. Mocuta, Boise, ID (US)

(21) Appl. No.: 17/720,770

(22) Filed: Apr. 14, 2022

(51) Int. Cl. A61B 5/18; A61B 5/00; B60R 25/25; B60W 60/00; G16H 40/63

(52) U.S. Cl. CPC A61B 5/18; A61B 5/7267; A61B 5/0022; A61B 5/6893; B60R 25/25; B60W 60/0051; G16H 40/63; B60W 2556/65; B60W 2540/221; B60W 2540/223; B60W 2540/225; B60W 2540/21

(57) ABSTRACT

Methods, devices, and systems related to determining driver capability are described. In an example, a method can include receiving, at a computing device, data associated with a driver from a sensor, inputting the data into an artificial intelligence (AI) model, performing an AI operation using the AI model, and determining whether the driver is capable of driving a vehicle based on an output of the AI model.

### TECHNICAL FIELD

The present disclosure relates generally to determining a capability of a driver.

### BACKGROUND

A vehicle can include one or more sensors. Operations can be performed based on data collected by the one or more sensors. For example, the vehicle can notify a driver of the vehicle that the vehicle is low on oil or gas.

A computing device can include a mobile device (e.g., a smart phone), a medical device, or a wearable device, for example. Computing devices can also include one or more sensors and perform operations based on data collected by the one or more sensors. For example, some computing devices can detect and store your location.

### BRIEF DESCRIPTION OF THE DRAWINGS

FIG. 1 illustrates an example of a computing device in accordance with a number of embodiments of the present disclosure.

FIG. 2 illustrates an example of a vehicle in accordance with a number of embodiments of the present disclosure.

FIG. 3 illustrates an example of a system including a computing device and a vehicle in accordance with a number of embodiments of the present disclosure.

FIG. 4 is a flow diagram of a method for determining driver capability in accordance with a number of embodiments of the present disclosure.

### DETAILED DESCRIPTION

The present disclosure includes methods, apparatuses, and systems related to determining driver capability. An example method includes receiving, at a computing device, data associated with a driver from a sensor, inputting the data into an artificial intelligence (AI) model, performing an AI operation using the AI model, and determining whether the driver is capable of driving a vehicle based on an output of the AI model.

People who suffer from re-occurring and intermittent health conditions may not be able to operate vehicles for fear of temporary impairment while driving. Re-occurring and intermittent health conditions can include, but are not limited to, vertigo, seizures, heart attacks, strokes, sleepiness, diabetes, and/or panic attacks. Temporary impairment could include dizziness, erratic body movement, uncoordinated movement, and/or loss of consciousness, for example. By collecting data on a driver and imputing the data into an AI model, the AI model can determine characteristics indicative of impairment events. Accordingly, the AI model can determine when a driver is incapable of driving prior to and/or while driving. This could enable people who suffer from reoccurring and intermittent health conditions to drive while reducing the risk of loss of life, injury, or property damage as a result of an accident due to an impairment event.

The data associated with the driver can include a heart rate, blood oxygen level, blood glucose level, blood pressure level, perspiration rate, respiration rate, electroencephalogram (EEG), electrocardiogram (EKG), electrooculogram (EOG), Electromyography (EMG), movement, temperature, facial color, facial expression, body language, eyelid coverage of an eye, eye blink frequency, eye color, eye dilation, eye direction, and/or voice of the driver. The data associated with the driver can be recorded by a heart rate monitor, a blood glucose monitor, an accelerometer, a gyroscope, a proximity sensor, a microphone, a camera, and/or a thermometer, for example. In a number of embodiments, the data associated with the driver can include a pressure applied to a steering wheel of the vehicle recorded by a pressure sensor of the vehicle and/or a driving assessment of the driver including the driver's ability to stay within a lane recorded by a camera on the vehicle. The sensor can be one of a number of sensors coupled to or included in the vehicle or the computing device.

The AI model can be trained outside of the vehicle and/or the computing device. For example, a cloud computing system can train the AI model with generic data and send the trained AI to the vehicle and/or a computing device. The vehicle and/or the computing device can store the AI model in a memory device. In some examples, the trained AI model can be updated periodically or in response to new generic data and/or specific driver data being used to train the AI model. A processing resource can receive the trained AI model directly from a cloud computing system or a memory device.

AI operations can be performed on the driver data using the AI model to determine whether the driver is capable of driving. The processing resource can include components configured to perform AI operations. In some examples, AI operations can include machine learning or neural network operations, which may include training operations or inference operations, or both.

One or more commands can be generated, sent, and/or executed in response to an output of the AI model. The commands can be sent to and/or executed by the computing device and/or the vehicle. Commands can include instructions to provide information, perform a function, or initiate autonomous driving of the vehicle, for example.

As used herein, “a number of” something can refer to one or more of such things. For example, a number of computing devices can refer to one or more computing devices. A “plurality” of something intends two or more.

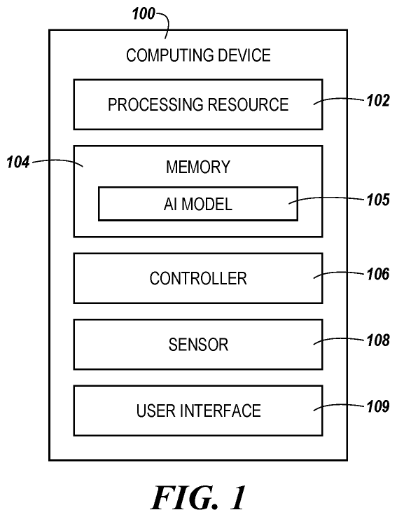

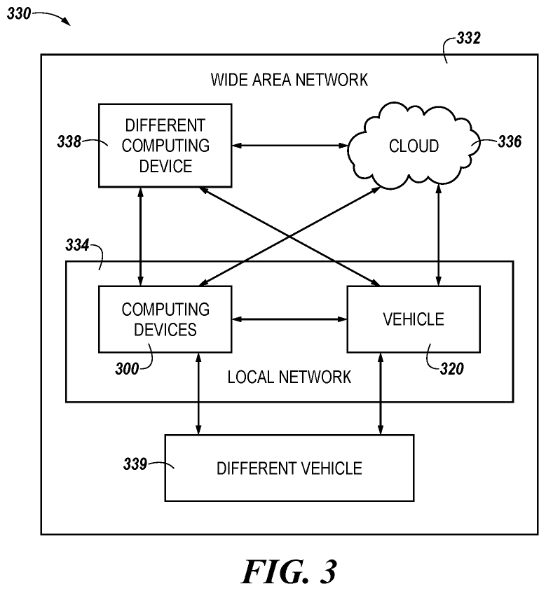

The figures herein follow a numbering convention in which the first digit or digits correspond to the drawing figure number and the remaining digits identify an element or component in the drawing. Similar elements or components between different figures may be identified by the use of similar digits. For example, reference numeral 100 may reference element “0” in FIG. 1, and a similar element may be referenced as 300 in FIG. 3. As will be appreciated, elements shown in the various embodiments herein can be added, exchanged, and/or eliminated so as to provide a number of additional embodiments of the present disclosure. In addition, the proportion and the relative scale of the elements provided in the figures are intended to illustrate various embodiments of the present disclosure and are not to be used in a limiting sense.

FIG. 1 illustrates an example of a computing device 100 in accordance with a number of embodiments of the present disclosure. The computing device 100 can be, but is not limited to, a wearable device, a medical device, and/or a mobile device. The computing device 100, as illustrated in FIG. 1, can include a processing resource 102, a memory 104 including an AI model 105, a controller 106, one or more sensors 108, and a user interface 109.

The memory 104 can be volatile or nonvolatile memory. The memory 104 can also be removable (e.g., portable) memory, or non-removable (e.g., internal) memory. For example, the memory 104 can be random access memory (RAM) (e.g., dynamic random access memory (DRAM) and/or phase change random access memory (PCRAM)), read-only memory (ROM) (e.g., electrically erasable programmable read-only memory (EEPROM) and/or compact-disc read-only memory (CD-ROM)), flash memory, a laser disc, a digital versatile disc (DVD) or other optical storage, and/or a magnetic medium such as magnetic cassettes, tapes, or disks, among other types of memory.

Further, although memory 104 is illustrated as being located within computing device 100, embodiments of the present disclosure are not so limited. For example, memory 104 can be located on an external apparatus (e.g., enabling computer readable instructions to be downloaded over the Internet or another wired or wireless connection).

Memory 104 can be any type of storage medium that can be accessed by the processing resource 102 to perform various examples of the present disclosure. For example, the memory 104 can be a non-transitory computer readable medium having computer readable instructions (e.g., computer program instructions) stored thereon that are executable by the processing resource 102 to receive data associated with a driver located in a vehicle from a sensor 108, input the data associated with the driver into an AI model 105, perform an AI operation using the AI model 105, and generate and/or send a command in response to an output of the AI model 105.

The AI model 105 can be trained outside of the computing device 100. For example, a cloud computing system (e.g., cloud computing system 336 in FIG. 3) can train the AI model 105 with generic data and send the AI model 105 to the computing device 100. For example, the AI model 105 can be trained with data from people who suffer from the same re-occurring and intermittent health condition as the driver. The computing device 100 can store the AI model 105 in memory 104 of the computing device 100.

In some examples, the AI model 105 can be updated and/or replaced periodically and/or in response to new data being available to train the AI model 105. For example, the AI model 105 can be updated with new clinical data and/or data associated with the driver including data indicative of a driver's baseline and/or data indicative of a driver just prior to an impairment event, during an impairment event, and/or just after an impairment event. Prior to an impairment event, a driver could begin closing their eyes for a longer than normal period of time and/or begin blinking rapidly. During an impairment event, a driver's eyes and/or head could be averted from the road and/or the driver's head could roll and/or jerk. After an impairment event, the driver could begin having their eyes open for a normal period of time, stop blinking rapidly, the driver's eyes and/or head could be directed towards the road, and/or the driver's head could stop rolling and/or jerking.

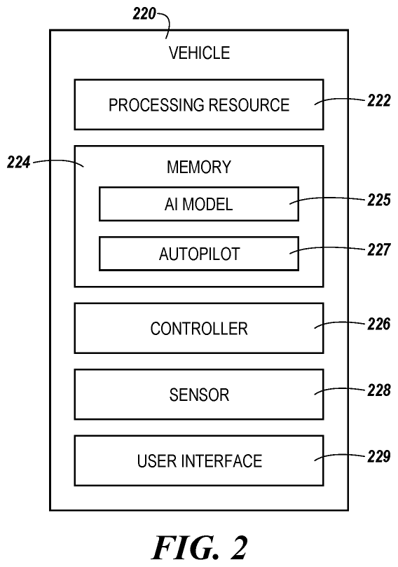

The processing resource 102 can receive the AI model 105 directly from a cloud computing system, memory 104, or memory (e.g., memory 224 in FIG. 2) of the vehicle. The processing resource 102 can also receive the data associated with the driver. The data associated with the driver can be collected from the one or more sensors 108 included in and/or coupled to the computing device 100 and/or the one or more sensors included in and/or coupled to the vehicle and can be stored in memory 104 and/or memory of the vehicle.

The one or more sensors 108 of the computing device 100 can collect data associated with the driver from a driver located outside of and/or within the vehicle. The one or more sensors 108 can detect a driver's movement, heart rate, blood oxygen level, blood glucose level, blood pressure level, perspiration rate, respiration rate, EEG, EKG, EOG, EMG, temperature, facial color, facial expression, body language, eyelid coverage, eye blink frequency, eye color, eye dilation, eye direction, and/or voice. The data associated with the driver can be recorded by a heart rate monitor, a blood glucose monitor, an accelerometer, a gyroscope, a proximity sensor, a microphone, a camera, and/or a thermometer, for example. In a number of embodiments, the data associated with the driver can include a pressure applied to a steering wheel of the vehicle recorded by a pressure sensor of the vehicle and/or a driving assessment of the driver including the driver's ability to stay within a lane recorded by a camera on the vehicle. The sensor can be one of a number of sensors coupled to or included in the vehicle or the computing device 100.

The computing device 100 can receive different data from applications and/or files located on the computing device 100, on the vehicle, and/or on a remote server, for example. The different data can include a dietary record, a sleep record, or a symptom record of the driver. In a number of embodiments, the different data can be weather data when an impairment event can be triggered by particular weather conditions.

AI operations can be performed on the data associated with the driver provided by the one or more sensors 108 and/or the different data from applications and/or files using the AI model 105. The processing resource 102 can include components configured to perform AI operations. In some examples, AI operations can include machine learning or neural network operations, which may include training operations or inference operations, or both. The processing resource 102 can provide an output of the AI model 105.

The controller 106 can generate one or more commands in response to the output of the AI model 105. The one or more commands can include instructions to provide information, generate a message, perform a function, and/or initiate autonomous driving of the vehicle. The controller 106 can send the one or more commands to the computing device 100, the vehicle, a different computing device, and/or a different vehicle.

The computing device 100 can execute the one or more commands. Execution of the one or more commands can include generating a message providing information to a driver located outside of or inside the vehicle. For example, instructions not to drive, to pull over, data associated with the driver, or directions to a nearest hospital or a safe parking spot could be provided.

The information and/or message can be provided via user interface 109. The user interface 109 can be generated by computing device 100 in response to one or more commands from controller 106. The user interface 109 can be a graphical user interface (GUI) that can provide and/or receive information to and/or from the user of the computing device 100. In a number of embodiments, the user interface 109 can be shown on a display of the computing device 100. For example, the user interface 109 can display a message that the driver is incapable of driving when the AI model 105 determines the driver is incapable of driving and/or the user interface 109 can display a message that the driver is capable of driving when the AI model 105 determines the driver is capable of driving.

In some examples, a message and/or information could be generated and transmitted to a different computing device, and/or different vehicle when the AI model 105 determines the driver is incapable of driving. For example, a location of the vehicle, audio, streaming audio, video, streaming video, data from one or more sensors 108 of the computing device 100, data from one or more sensors of the vehicle, a medical report of a driver outside or inside the vehicle, and/or a condition of the vehicle could be sent to an emergency contact or an emergency service provider (e.g., hospital, police, fire department, mechanic, tow company) via the computing device 100.

In a number of embodiments, the computing device 100 can open a particular application when the AI model 105 determines the driver is incapable of driving. For example, the computing device 100 can ride-hail a car (e.g., hire a car service to take them to a particular destination) using an application on the computing device 100 and the location of the computing device 100.

In some examples, the AI model 105 can determine the driver is capable of driving for a particular period of time. For example, if the driver is not currently showing any advance signs of an impairment event, the AI model 105 can determine the driver is capable of driving for the amount of time it takes between the start of advance signs and an impairment event. The computing device 100 could transmit a command to the vehicle to allow the driver to drive the vehicle during the particular time period in some instances.

FIG. 2 illustrates an example of a vehicle 220 in accordance with a number of embodiments of the present disclosure. The vehicle 220 can be, but is not limited to, a human operated vehicle, a self-driving vehicle, or a fully autonomous vehicle. The vehicle 220, as illustrated in FIG. 2, can include a processing resource 222, a memory 224 including an AI model 225 and an autopilot 227, a controller 226, one or more sensors 228, and a user interface 229.

The memory 224 can be volatile or nonvolatile memory. Although memory 224 is illustrated as being located within vehicle 220, embodiments of the present disclosure are not so limited. For example, memory 224 can be located on an external apparatus.

Memory 224 can be any type of storage medium that can be accessed by the processing resource 222 to perform various examples of the present disclosure. For example, the memory 224 can be a non-transitory computer readable medium having computer readable instructions stored thereon that are executable by the processing resource 222 to receive data associated with a driver located in the vehicle 220 from the sensor 228, input the data associated with the driver into the AI model 225, and generate and/or send a command in response to an output of the AI model 225.

The AI model 225 can be trained outside of the vehicle 220. For example, a cloud computing system (e.g., cloud computing system 336 in FIG. 3) can train the AI model 225 with generic data and send the AI model 225 to the vehicle 220. The vehicle 220 can store the AI model 225 in memory 224 of the vehicle 220 and/or memory (e.g., memory 104 in FIG. 1) of the computing device.

In some examples, the AI model 225 can be updated and/or replaced periodically or in response to new data being available to train the AI model 225. For example, the AI model 105 can be updated with new clinical data and/or data associated with the driver including data indicative of a driver's baseline and/or data indicative of a driver just prior to an impairment event, during an impairment event, and/or just after an impairment event.

The processing resource 222 can receive the AI model 225 directly from a cloud computing system, memory 224 of the vehicle 220, or the memory of the computing device. The processing resource 222 can also receive data associated with the driver. The data associated with the driver can be collected from the one or more sensors included in and/or coupled to the computing device or the one or more sensors 228 included in and/or coupled to the vehicle 220 and can be stored in memory 224 of the vehicle 220 and/or memory of the computing device.

The one or more sensors 228 of the vehicle 220 can collect data associated with the driver located outside of and/or within the vehicle. The one or more sensors 228 can detect a driver's movement, heart rate, blood oxygen level, blood glucose level, blood pressure level, perspiration rate, respiration rate, EEG, EKG, EOG, EMG, temperature, facial color, facial expression, body language, eyelid coverage, eye blink frequency, eye color, eye dilation, eye direction, and/or voice. The data associated with the driver can be recorded by a heart rate monitor, a blood glucose monitor, an accelerometer, gyroscope, a proximity sensor, a microphone, a camera, and/or a thermometer, for example. In a number of embodiments, the data associated with the driver can include a pressure applied to a steering wheel of the vehicle 220 recorded by a pressure sensor of the vehicle 220 and/or a driving assessment of the driver including the driver's ability to stay within a lane recorded by a camera on the vehicle 220. The one or more sensors 228 can also collect data associated with the vehicle 220, for example, the one or more sensors 228 can detect a location, speed, surroundings, traffic, traffic signs, traffic lights, and/or state of the vehicle 220.

The vehicle 220 can receive different data from applications and/or files located on the vehicle 200, the computing device, and/or on a remote server, for example. The different data can include a dietary record, a sleep record, or a symptom record of the driver. In a number of embodiments, the different data can be weather data when an impairment event can be triggered by particular weather conditions.

AI operations can be performed on the data from the one or more sensors included in and/or coupled to the computing device and/or the one or more sensors 228 included in and/or coupled to the vehicle 220 using the AI model 225. The processing resource 222 can include components configured to perform AI operations. In some examples, AI operations can include machine learning or neural network operations, which may include training operations or inference operations, or both. The processing resource 222 can provide an output of the AI model 225.

The controller 226 can generate one or more commands in response to the output of the AI model 225. The one or more commands can include instructions to provide information, generate a message, perform a function, and/or initiate autonomous driving of the vehicle 220. The controller 226 can send the one or more commands to the computing device, the vehicle 220, and/or a different vehicle (e.g., different vehicle).

The vehicle 220 can execute the one or more commands. Execution of the one or more commands can include generating a message providing information to a driver located outside of or inside the vehicle 220. For example, instructions not to drive, to pull over, data associated with the driver, or directions to a nearest hospital or a safe parking spot could be provided.

The information can be provided via user interface 229, for example. The user interface 229 can be generated by vehicle 220 in response to one or more commands from controller 226. The user interface 229 can be a GUI that can provide and/or receive information to and/or from the driver of the vehicle 220. In a number of embodiments, the user interface 229 can be shown on a display of the vehicle 220. For example, the user interface 229 can display a message that the driver is incapable of driving when the AI model 105 determines the driver is incapable of driving and/or the user interface 229 can display a message that the driver is capable of driving when the AI model 225 determines the driver is capable of driving.

In some examples, a message and/or information could be generated and transmitted to the computing device, a different computing device, and/or different vehicle when the AI model 225 determines the driver is incapable of driving. For example, a location of the vehicle 220, audio, streaming audio, video, streaming video, data from one or more sensors 228 of the vehicle 220, data from one or more sensors of the computing device, a medical report of a driver outside or inside the vehicle, and/or a condition of a vehicle could be sent to an emergency contact and/or an emergency service provider (e.g., hospital, police, fire department, mechanic, tow company) via the vehicle 220.

The vehicle 220 can perform one or more functions in response to the one or more commands from the controller 226. For example, the processing resource 222 could establish that the driver is showing characteristics indicative of an impending and/or current impairment event and determine the driver is or soon will be incapable of driving the vehicle 220. In response to this determination, the controller 226 can generate and/or send a command to the vehicle 220 to, for example, lock the vehicle 220 to prevent the driver from entering the vehicle 220, disable movement of the vehicle 220 to prevent the driver from driving the vehicle 220, display a message to notify the driver not to drive or to pull over the vehicle 220, open a particular application on the computing device, turn on hazard lights, engage an emergency brake, turn off the engine, and/or initiate autopilot 227 of the vehicle 220. The autopilot 227 can enable the vehicle 220 to self-drive or be fully autonomous. Opening the particular application could be a ride hailing application, for example.

In some examples, the AI model 225 can determine the driver is capable of driving for a particular period of time. For example, if the driver is not currently showing any advance signs of an impairment event, the AI model 225 can determine the driver is capable of driving for the amount of time it takes between the start of advance signs and an impairment event. The vehicle 220 could allow the driver to drive the vehicle 220 during the particular time period.

FIG. 3 illustrates an example of a system 330 including a computing device 300 and a vehicle 320 in accordance with a number of embodiments of the present disclosure. Computing device 300 can correspond to computing device 100 in FIG. 1 and vehicle 320 can correspond to vehicle 220 in FIG. 2. The system 330 can include a wide area network (WAN) 332 and a local area network (LAN) 334. The LAN 334 can include the computing device 300 and the vehicle 320. The WAN 332 can further include a cloud computing system 336, a different computing device 338, and a different vehicle 339.

The WAN 332 can be a distributed computing environment, the Internet, for example, and can include a number of servers that receive information from and transmit information to the cloud computing system 336, the different computing device 338, the computing device 300, the vehicle 320, and/or the different vehicle 339. Memory and processing resources can be included in the cloud computing system 336 to perform operations on data. The cloud computing system 336 can receive and transmit information to the different computing device 338, the computing device 300, the vehicle 320, and/or the different vehicle 339 using the WAN 332. As previously described, the computing device 300 and/or the vehicle 320 can receive an AI model from cloud computing system 336.

The cloud computing system 336 can train the AI model with generic data. The generic data can be data from studies of re-occurring and intermittent health conditions and/or manufacturers of the one or more sensors, the computing device 300, and/or the vehicle 320. For example, the generic data can be data collected from a manufacturer's in field testing. In some examples, the generic data can be collected from different computing devices and/or vehicles.

The LAN 334 can be a secure (e.g., restricted) network for communication between the computing device 300 and the vehicle 320. The LAN 334 can include a personal area network (PAN), for example Bluetooth or Wi-Fi Direct. In some examples, a number of computing devices within or within a particular distance of the vehicle 320 can transmit and/or receive data via LAN 334. The sensor data from the computing device 300 and/or the vehicle 320 can be solely used for AI operations within the LAN 334 to protect driver data from theft. For example, sensor data from computing device 300 and/or vehicle 320 will not be used and/or transmitted outside of the LAN 334 unless permitted by the user of the computing device 300 and/or the vehicle 320.

In a number of embodiments, data can be transmitted to the different computing device 338 and/or the different vehicle 339 via WAN 332 in response to a command from the computing device 300 and/or the vehicle 320. The different computing device 338 could be a computer, a wearable device, or a mobile device of an emergency contact set by the driver or an emergency service provider (e.g., hospital, police, fire department, mechanic, tow company), for example. Data sent to the different computing device 338 located outside of the vehicle 320 and/or the different vehicle 339 could provide a location of the vehicle 320, audio, streaming audio, video, streaming video, data from one or more sensors, a medical report of a person outside or inside the vehicle 320, a condition of the vehicle 320, and/or a command. For example, a command could be transmitted to the different vehicle 339. The different vehicle 339 could receive the command and notify a driver of the different vehicle or initiate autopilot of the different vehicle 339 to avoid the vehicle 320.

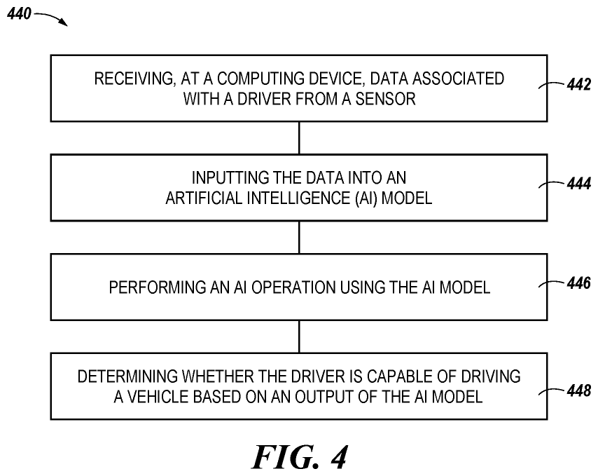

FIG. 4 is a flow diagram of a method 440 for determining driver capability in accordance with a number of embodiments of the present disclosure. At block 442, the method 440 can include receiving, at a computing device, data associated with a driver from a sensor. The data associated with the driver can include a heart rate, blood oxygen level, blood glucose level, blood pressure level, perspiration rate, respiration rate, EEG, EKG, EOG, EMG, temperature, facial color, facial expression, body language, eyelid coverage, eye blink frequency, eye color, eye dilation, eye direction, or voice of the driver. The sensor can be coupled to or included in a vehicle or a computing device including a mobile device, a medical device, or a wearable device.

At block 444, the method 440 can include inputting the data into an AI model. The AI model can be trained with clinical data and/or data from people who suffer from the same re-occurring and intermittent health condition as the driver. The AI model can also be trained with data associated with the driver. The data associated with the driver can enable the AI model to establish normal characteristics of the driver, characteristics of the driver just prior to an impairment event, characteristics of the driver during an impairment event, and/or characteristics of the driver just after an impairment event.

At block 446, the method 440 can include performing an AI operation using the AI model. A processing resource can include components configured to perform AI operations. In some examples, AI operations can include machine learning or neural network operations, which may include training operations or inference operations, or both.

At block 448, the method 440 can include determining whether the driver is capable of driving a vehicle based on an output of the AI model. The AI model may determine the driver is incapable of driving in response to establishing that the driver is showing characteristics indicative of an impairment event or the AI model may determine the driver is capable of driving in response to establishing that the driver is not showing any characteristics indicative of an impairment event.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and methods are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A method, comprising:
receiving, at a computing device, data associated with a driver from a sensor;
inputting the data into an artificial intelligence (AI) model;
performing an AI operation using the AI model; and
determining whether the driver is capable of driving a vehicle based on an output of the AI model.

2. The method of claim 1, wherein the data associated with the driver includes at least one of a heart rate, blood oxygen level, blood glucose level, blood pressure level, perspiration rate, respiration rate, electroencephalogram (EEG), electrocardiogram (EKG), electrooculogram (EOG), Electromyography (EMG), movement, temperature, facial color, facial expression, body language, eyelid coverage, eye blink frequency, eye color, eye dilation, eye direction, or voice of the driver.

3. The method of claim 1, further comprising:
receiving different data including at least one of a dietary record, a sleep record, or a symptom record of the driver;
inputting the different data into the AI model;
performing the AI operation using the AI model; and
determining whether the driver is capable of driving the vehicle based on the output of the AI model.

4. The method of claim 1, further comprising:
receiving weather data; and
inputting the weather data into the AI model;
performing the AI operation using the AI model; and
determining whether the driver is capable of driving the vehicle based on the output of the AI model.

5. The method of claim 1, further comprising:
generating a message in response to determining the driver is incapable of driving the vehicle; and
displaying the message on a user interface of the computing device in response to generating the message.

6. The method of claim 1, further comprising:
generating a message in response to determining the driver is incapable of driving the vehicle; and
transmitting the message to a different computing device in response to generating the message.

7. The method of claim 1, further comprising opening a particular application on the computing device in response to determining the driver is incapable of driving the vehicle.

8. The method of claim 1, further comprising:
generating a command in response to determining the driver is incapable of driving the vehicle;
transmitting the command to the vehicle in response to generating the command;
receiving the command at the vehicle; and
locking the vehicle in response to receiving the command.

9. The method of claim 1, further comprising:
determining the driver is capable of driving the vehicle for a particular time period based on the output of the AI model;
generating a message in response to determining the driver is capable of driving the vehicle for the particular time period; and
displaying the message on a user interface of the computing device in response to generating the message.

10. The method of claim 1, further comprising:
determining the driver is capable of driving the vehicle for a particular time period based on the output of the AI model;
generating a command in response to determining the driver is capable of driving for the particular time period;
receiving the command at the vehicle; and
allowing the driver to drive the vehicle during the particular time period.

11. An apparatus, comprising:
a processing resource configured to:
receive data associated with a driver located in a vehicle from a sensor;
input the data associated with the driver into an artificial intelligence (AI) model; and
perform an AI operation using the AI model; and
a controller configured to:
send a command in response to an output of the AI model.

12. The apparatus of claim 11, wherein the apparatus is the vehicle or a computing device.

13. The apparatus of claim 11, further comprising a memory device configured to store at least one of the trained AI model or the data associated with the driver.

14. The apparatus of claim 11, wherein the AI model is received from a cloud computing system.

15. The apparatus of claim 11, wherein the sensor is included in a wearable device, a medical device, a mobile device, or the vehicle.

16. A system, comprising:
a sensor; and
a vehicle including:
a processing resource configured to:
receive data associated with a driver located in the vehicle from the sensor;
input the data associated with the driver into an artificial intelligence (AI) model; and
perform an AI operation using the AI model; and
a controller configured to send a command in response to an output of the AI model.

17. The system of claim 16, wherein the command initiates autopilot for the vehicle.

18. The system of claim 16, further comprising a different vehicle, wherein the controller is configured to send the command to the different vehicle in response to the output of the AI model.

19. The system of claim 18, wherein the different vehicle is configured to:
receive the command; and
notify a different driver of the different vehicle of the vehicle.

20. The system of claim 18, wherein the different vehicle is configured to:
receive the command; and
initiate autopilot of the different vehicle.
